(19) United States
(12) Patent Application Publication
(10) Pub. No.: US 2020/0324147 A1
(43) Pub. Date: Oct. 15, 2020

(54) A Pain Relief, Fracture Healing and Spinal Fusion Device

(71) Applicants: Quanqin Dai, Diamond Bar, CA (US); Trevor Theriot, Las Vegas, NV (US); John Lasso, Lagina Hills, CA (US); Joseph Horton, Newport Beach, CA (US)

(72) Inventors: Quanqin Dai, Diamond Bar, CA (US); Trevor Theriot, Las Vegas, NV (US); John Lasso, Lagina Hills, CA (US); Joseph Horton, Newport Beach, CA (US)

(21) Appl. No.: 16/380,913

(22) Filed: Apr. 10, 2019

(51) Int. Cl. A61N 7/02; A61N 1/04

(52) U.S. Cl. CPC A61N 7/02; A61N 2007/0026; A61N 1/0456

(57) ABSTRACT

A pain relief, fracture healing and spinal fusion device that comprises of a carrier that defines a centrally placed ultrasound transducer receiving hub and a left and a right electrode sheet, an ultrasound transducer that attaches to the receiving hub, and a controlled electrical generating device that connects to the carrier. The controlled electrical generating device powers and controls the frequency of the ultrasound transducer and the current delivered to the electrode sheets.

### BACKGROUND

The present invention is directed to a pain relief, fracture Healing and spinal fusion device.

Fracture healing involves a complex interplay of cellular processes, culminating in bridging of a fracture gap with bone.¹ Low-intensity pulsed ultrasound (LIPUS) has been proposed as a modality that may have a benefit for increasing reliable fracture healing as well as perhaps increasing the rate of fracture healing.² ¹ Available at https://www.ncbi.nlm.nih.gov/pmc/articles/PMC2748418/, Della Rocca G J. The science of ultrasound therapy for fracture healing. Indian J Orthop. 2009; 43(2):121-126. doi:10.4103/0019-5413.50845, seen on Apr. 9, 2019.² Id.

Low-intensity pulsed ultrasound (LIPUS) is a relatively new technique for the acceleration of fracture healing in fresh fractures and nonunions.³ It has a frequency of 1.5 MHz, a signal burst width of 200 μs, a signal repetition frequency of 1 kHz, and an intensity of 30 mW/cm2.⁴ ³ Available at https://journals.lww.com/jorthotrauma/fulltext/2010/03001/Ultrasound for Fracture Healing Current Evidence.12.aspx, Ultrasound for Fracture Healing: Current Evidence, Watanabe, Yoshinobu M D, PhD*; Matsushita, Takashi M D, DMSc*; Bhandari, Mohit M D, MSc, FRCSC†; Zdero, Rad PhD‡§; Schemitsch, Emil H M D, FRCSC‡∥, Journal of Orthopaedic Trauma: March 2010—Volume 24—Issue—p S56-S61, seen on Apr. 10, 2019.⁴ Id.

Physical therapist use ultrasound therapy to heal injuries. Unfocused beams of ultrasound for physical therapy were the first clinical application, dating to the 1950s, which often has been referred to simply as “therapeutic ultrasound” (Robertson and Baker, 2001).⁵ This modality now typically has a base unit for generating an electrical signal and a hand-held transducer.⁶ The hand-held transducer is applied with coupling gel and moved in a circular motion over an injured or painful area of the anatomy to treat conditions such as bursitis of the shoulder or tendonitis, by trained physical therapy technicians.⁷ The objective is to warm tendons, muscle and other tissue to improve blood flow and accelerate healing.⁸ ⁵ Available at https://www.ncbi.nlm.nih.gov/pmc/articles/PMC3810427/, Miller D L, Smith N B, Bailey M R, et al. Overview of therapeutic ultrasound applications and safety considerations. J Ultrasound Med. 2012; 31(4):623-634. Seen on Apr. 10, 2019.⁶ Id.⁷ Id.⁸ Id.

Therapy involves multiple treatments of 20 min each day by applying the large flat transducer to the site of injury and continuing treatment for periods of months.⁹ Although the process appears to be safe and effective, the therapy is slow and its use is predominantly limited to management of non-healing fractures.¹⁰ ⁹ Id.¹⁰ Id.

Transcutaneous electrical nerve stimulation (TENS) is a commonly used nonpharmacologic and noninvasive treatment for pain.¹¹ A TENS device is used to provide the electrical nerve stimulation. TENS is the application of electrical current through electrodes placed on the skin for pain control. It can be applied with varying frequencies, from low (<10 Hz) to high (>50 Hz).¹² Regardless of intensity, different frequencies activate central mechanisms to produce analgesia.¹³ Low-frequency TENS activates μ-opioid receptors in the spinal cord and the brainstem, whereas high-frequency TENS activates δ-opioid receptors in the spinal cord and the brainstem.¹⁴ Basic scientific evidence suggests that there are peripheral and central nervous system mechanisms underlying the analgesic action of TENS.¹⁵ ¹¹ Available at https://www.ncbi.nlm.nih.gov/pmc/articles/PMC2746624/, DeSantana J M, Walsh D M, Vance C, Rakel B A, Sluka K A. Effectiveness of transcutaneous electrical nerve stimulation for treatment of hyperalgesia and pain. Curr Rheumatol Rep. 2008; 10(6):492-499. Seen on Apr. 10, 2019.¹² Id.¹³ Id.¹⁴ Id.¹⁵ Id.

The present invention provides a device that delivers an ultrasound treatment to a fracture while simultaneously delivering an electric nerve stimulation treatment to the areas that surround the fracture. The ultrasound will aid in the healing of the fracture and the electric nerve stimulation will reduce the pain surrounding the fracture and increase the blood flow for the healing of the fracture.

Low-intensity pulsed ultrasound therapy may be a useful means to ensure successful spine fusion.¹⁶ ¹⁶ Available at http://www.sciencedirect.com/science/article/pii/S1529943001000869, Low-intensity pulsed ultrasound improves spinal fusion, by Stephen D. Cook, Samantha L. Salkeld, Mse, Laura Popich Patron, John P. Ryaby, Thomas S. Whitecloud, The Spine Journal, July-August 2001, last seen on Apr. 10, 2019.

For the foregoing reason there is a need for a pain relief, fracture healing and spinal fusion device.

### SUMMARY

The present invention describes a pain relief, fracture healing and spinal fusion device.

The pain relief, fracture healing and spinal fusion device comprises of a carrier that has a centrally placed ultrasound transducer and a left and a right electrode sheet, and a controlled electrical generating device that connects to the electrode sheets and the ultrasound transducer, the controlled electrical generating device powers and controls the ultrasound transducer and the electrode sheets.

The electrode sheets are covered with an electrically conductive gel that may be comprised of pain relieving analgesic.

An object of the present invention is to provide a device that will aid in the healing of fractures.

Another object of the present invention is to provide a device that will reduce pain around areas that surround a fracture.

Yet another object of the present invention is to provide a device that will increase the blood flow around the areas being treated by the device.

Still another object of the present invention is to provide a handheld device that will be used to reduce pain.

Yet still another object of the present invention is to provide a handheld device that can be used by a user to heal a fracture.

### DRAWINGS

These and other features, aspects, and advantages of the present invention will become better understood with regards to the following description, appended claims, and drawings where:

FIG. 1 is a photograph that shows all of the elements of the present invention;

FIG. 2 is a photograph that shows the carrier of the present invention and the electrode sheet that is applied to a user of the device;

FIG. 3 is a photograph that shows the ultrasound transducer of the present invention attached to the ultrasound transducer receiving hub of the carrier; and

FIG. 4. is a photograph of the controlled electrical generating device.

### DESCRIPTION

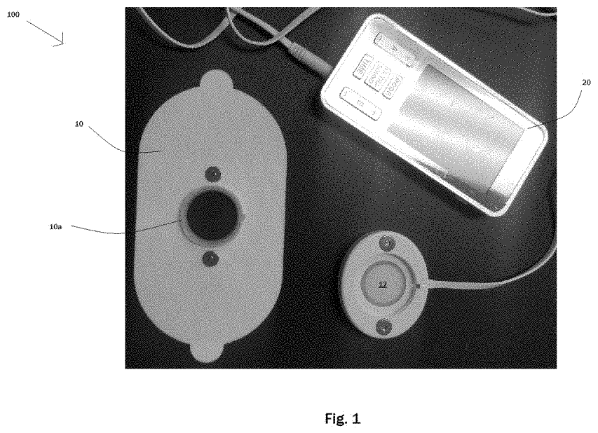

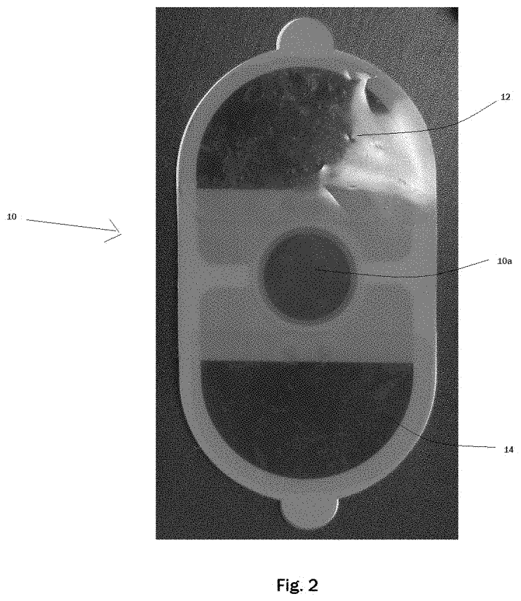

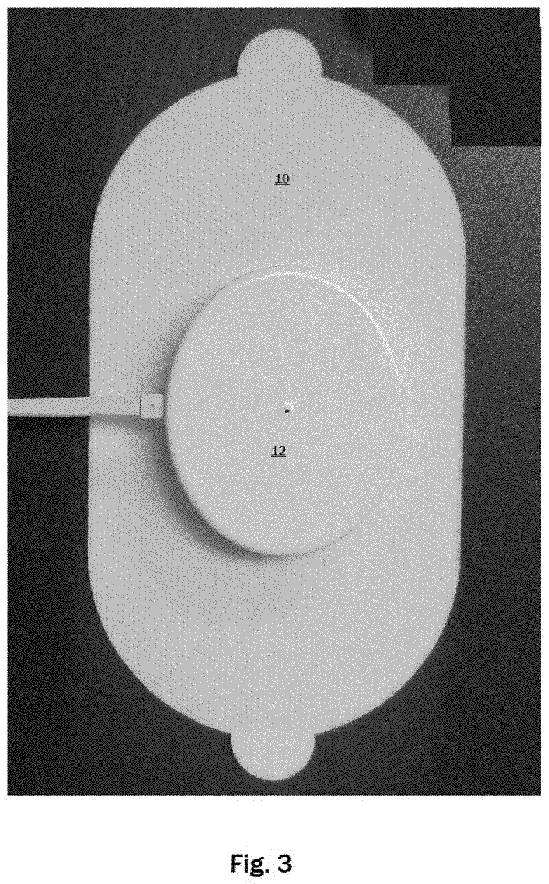

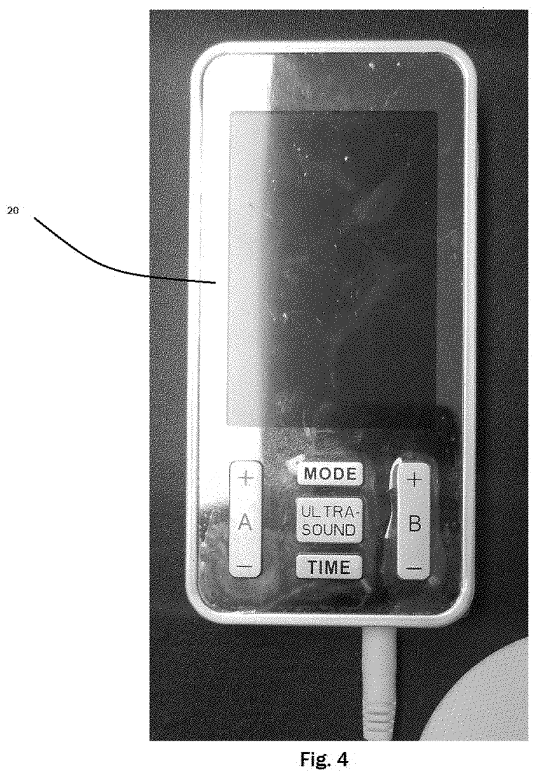

As seen in FIGS. 1-4, the present invention describes a pain relief, fracture healing and spinal fusion device 100. The pain relief, fracture healing and spinal fusion device 100 comprises of a carrier 10 that has a centrally placed ultrasound transducer receiving hub 10a and a left 14 and a right electrode sheet 16, an ultrasound transducer 12 that attaches to the ultrasound receiving hub 10a and a controlled electrical generating device 20 that connects to the electrode sheets 14-16 and the ultrasound transducer 12, the controlled electrical generating device 20 powers and controls the frequency of the ultrasound transducer 12 and the current delivered to the electrode sheets 14 and 16.

In an embodiment of the present invention, the electrode sheets 14 and 16 are covered with an electrically conductive gel. In a further embodiment of the invention, the conductive gel comprises of at least one pain relieving analgesic. The at least one pain relieving analgesic is selected from the group consisting of a Menthol, a CBD, a Lidocaine or a Capsaicin.

The pain relief and fracture healing device 100 is used by placing the carrier 10 over the area that is to be treated and then powering the controlled electric generating device 20 and lastly adjusting the frequency that will be delivered by the ultrasound transducer 12 and the current that will be delivered to the electrode sheets 14 and 16. The device can be used for 20 minute intervals on a daily basis.

An advantage of the present invention is that it provides a device that aids in the healing of fractures.

Another advantage of the present invention is that it provides a device that reduces pain around areas that surround a fracture.

Yet another advantage of the present invention is that it provides a device that increases the blood flow around the areas being treated by the device.

Still another advantage of the present invention is that it provides a handheld device that is used to reduce pain.

Yet still another advantage of the present invention is that it provides a handheld device that is used by a user to heal a fracture.

While the inventor's above description contains many specificities, these should not be construed as limitations on the scope, but rather as an exemplification of several preferred embodiments thereof. Accordingly, the scope should not be determined by the embodiments illustrated, but by the appended claims and their legal equivalents.

What is claimed is:

1. A pain relief, fracture healing and spinal fusion device, the healing device comprises of:
a carrier that defines a centrally placed ultrasound transducer receiving hub and a left and a right electrode sheet;
an ultrasound transducer that attaches to the ultrasound transducer receiving hub; and
a controlled electrical generating device that connects to the left and right electrode sheets and to the ultrasound transducer, the controlled electrical generating device powers and controls the frequency of the ultrasound transducer and the current delivered to the electrode sheets.

2. The pain relief, fracture healing and spinal fusion device of claim 1, wherein the electrode sheets are covered with an electrically conductive gel.

3. The pain relief, fracture healing and spinal fusion device of claim 2, wherein the electrically conductive gel comprises of at least one pain relieving analgesic.

4. The pain relief, fracture healing and spinal fusion device of claim 3, wherein the at least one pain relieving analgesic is selected from the group consisting of a Menthol, a CBD, a Lidocaine or a Capsaicin.

5. The pain relief, fracture healing and spinal fusion device of claim 4, wherein the frequency is in the range from about 1.0 MHZ to about 5.0 MHZ.

6. The pain relief, fracture healing and spinal fusion device of claim 1, wherein the frequency is in the range from about 1.0 MHZ to about 5.0 MHZ.
